(19) United States
(12) Patent Application Publication
(10) Pub. No.: US 2011/0111500 A1
(43) Pub. Date: May 12, 2011

(54) 3-Dimensional feeders for single cell co-culture in microarray slides

(76) Inventor: Stephen Iiye Chen, El Monte, CA (US)

(21) Appl. No.: 12/590,464

(22) Filed: Nov. 9, 2009

(51) Int. Cl. C12N 5/00

(52) U.S. Cl. 435/373

(57) ABSTRACT

This invention provides a co-culture method using 3-dimensional feeders to support single cell in microwells of microarray chips. Microbeads are utilized as carrier to manipulate feeders into 3-dimensional layers in a microwell, to retain feeders at desired location, to keep feeders away from single cell at a desired distance, to revive feeders for optimized co-culture, and to eliminate feeders from image background in post imaging analysis.

### BACKGROUND OF THE INVENTION

1. Field of the Invention

The present invention relates to methods of cell culture. In particular, it relates to a co-culture method using feeders to enhance viability of hard to culture cells in microwells of microarray slides.

2. Description of Prior Art

Cell lines and primary cells can be cultured in vitro. A cell line is a pure population of derivative cells established for easy growth in vitro. Primary cells are obtained from animal or human tissues. The in vitro maintenance of primary cells is much harder than that of cell lines. To enhance their viability, a population of supportive cells, called feeders, is frequently required to condition the culture medium and stimulate cell growth, such as fibroblast cells to support stem cells and glial cells to support neurons.

To set up a co-culture between feeders and cells, a prior preparation of feeder plates is required. During the preparation, feeders attach and grow on bottom surface of plates while waiting for co-culture with primary cells. The condition of the feeders changes rapidly during the waiting period, which results in a very short usable time period of the plates. In practice, prepared feeder plates are frequently wasted when the scheduled primary cells are not available at the right timing as wish.

With the exploration of microarray technology, a single cell could be cultured in a microwell. The bottom diameter of a microwell becomes tiny small in 1 mm or less. If using traditional method to setup a co-culture, feeders will occupy the entire bottom instantly, which makes feeder supportive co-culture a challenge in microarray chips.

Attempts have been made to develop co-culture and cell microarray chips.

Mussi, et al, in U.S. Pat. No. 5,409,829, teaches a co-culture method using a molded plastic insert in a large well. Mussi, et al failed to explain how to reduce the size of the insert to micrometer scale for fitting into microarray chips.

Sara, Lindstrom et al. in a publication of PLoS One, 2009 Sep. 14: 4 (9), introduce a high-density microwell chip for culture and analysis of stem cells. 672 microwells were constructed on a microscopic slide. The size of 672 microwells is so small that they failed to include feeders into their chips. Without feeders, the application of their chips is limited within easy growing cells.

Colin Ingham et al. in a publication of Proc Natl Acad Sci USA, 2007 Nov. 13; 104 (46): 18217-22, introduce a high density chip containing one million microwells on a slide. They failed to include feeders into their system. Their one million-well chips were used for growth of bacteria instead of stem cells or neurons.

A single cell co-culture system with supportive feeders in microarray chips is highly desirable but remains unsolved.

### SUMMARY OF THE INVENTION

The present invention teaches an easy method to put feeders and a single cell into microwells of microarray chips. Microbeads are utilized as carriers to build a 3-dimensional feeders inside a microwell. The novelty of the invention shows significant advantages:
a. It saves animal. One stock of feeders can be used for long period in numerous co-cultures.
b. It saves plates or microarray chips. The requirement of preparing feeders in plates is omitted.
c. It creates continuous availability of feeders to fit any timing requirement of co-cultures.
d. It creates a novel method of reviving feeders back to optimized condition easily during co-culture.
e. It creates high quality image without background of feeders in post imaging analysis.

### DESCRIPTION OF THE DRAWINGS

FIG. 1 is a diagram showing a corner of a 50 ml culture flask in magnified view to illustrate how to create a continuous supply of feeders.

FIG. 2 is a diagram of a microwell of a microarray chip in magnified view illustrating the setting of a 3-dimensional feeders to support a single cell in co-culture.

### DETAILED DESCRIPTION OF THE INVENTION

The technology of making microbeads has been established. Microbeads can be made with a variety of water-insoluble materials into certain sizes, such as a diameter of 10 micrometers. The density of microbeads can be controlled. They are usually heavier than water if made as solid spheres. But they can be slightly lighter than water if made as hollow spheres with air trapped inside. Hollow microbeads, such as hollow glass microspheres, have been used as industrial raw material for construction. In pharmaceuticals, microbeads have been used in bioreactors to increase drug productivity in manufacture.

The essential idea of the invention is a combination of microbeads with microarray chips to create a 3-dimensional feeder supportive single cell co-culture system. The surface of microbeads can be coated with polylysine to enhance cell attachment. When two units of microbeads contact each other in culture medium, feeders from the first unit can expand onto the second unit. The diameter of microbeads is tiny small around 10 micrometers, which makes microbeads invisible by eye and moving freely through pipette tip in culture medium.

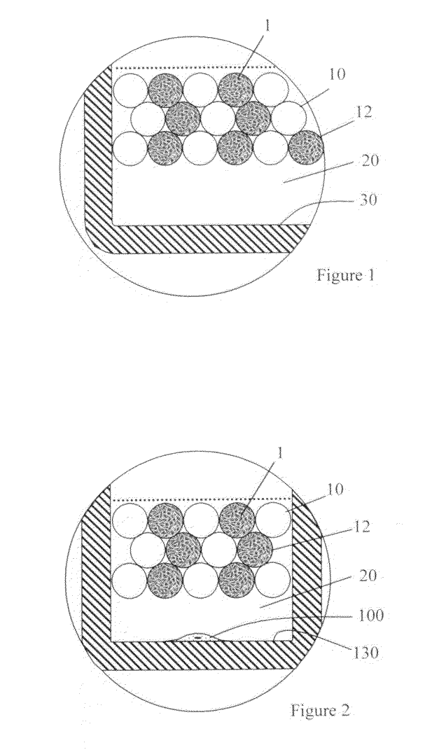

To establish an initial stock of feeders, the procedure is:
1. Use little culture medium 20 just enough to immerse microbeads 10 in a flask 30.
2. Add feeders 1 on top of microbeads 10 and incubate feeders 1 in a culture incubator for one hour. During the incubation feeders 1 attach to surface of microbeads 10.
3. Increase the volume of culture medium 20 in flask 30 and let feeders 1 to grow on surface of microbeads 10.
4. After 7 days in culture, most of microbeads 10 become occupied microbeads 12 covered by feeders 1.
5. To revive the growth of feeders 1, an equal amount of fresh microbeads 10 can be mixed into occupied microbeads 12 in flask 30, as shown in FIG. 1.
6. Put the feeders back to culture incubator for continuous growth.

In the embodiment, microbeads 10 are hollow microspheres with air trapped inside. They have slightly light density than culture medium 20 and float upwards if staying steady for a while. A change of old culture medium 20 can be done by deeply inserting a pipette tip to bottom of flask 30.

Microbeads 10 with attached feeders 1 in flask 30 is a universal stock of feeders 1. A universal stock is compatible for co-cultures in a variety of formats, such as 6-well plates, 96-well plates, 1536-well plates, or microarray chips. To transfer feeders 1 from flask 30 to a co-culture plate, culture medium 20 is gently swirled and a certain volume is transferred to the co-culture plate. Feeders 1 are transferred together with culture medium 20.

In prior art, trypsin, a protease, is required to digest cells and cause their detachment off bottom surface. One problem is that trypsin can cause abnormality of feeders. To avoid using trypsin, animals were sacrificed repeatedly in preparing fresh feeders each time for a co-culture. The present invention is a significant achievement in saving animal life. Trypsin is omitted. The stock of feeders can be continuously revived for numerous co-cultures without sacrificing animals repeatedly.

Cells like to stay together in culture dishes. In microarray chips, the viability of cells is dramatically reduced if a single cell is cultured alone. Feeder support becomes critical for hard to culture cells.

FIG. 2 shows a setting of a 3-dimensional feeders with a single cell in a microwell.
The co-culture procedure is:
1. Dilute cells in culture medium 20 to very low counts, such as 1 cell/ul.
2. Add 1 ul of culture medium 20 to a microwell 130 to make most of the microwells having a single cell respectively.
3. Incubate the cells in a culture incubator for 1 hour so that a single cell 100 attaches onto bottom of microwell 130.
4. Transfer 2 ul of swirled culture medium 20, containing microbeads 10 and attached feeders 1, from flask 30 into microwell 130 to co-culture with single cell 100, as shown in FIG. 2.
5. Incubate the co-culture for a desired time length until single cell 100 is ready for imaging.
6. For high quality imaging of single cell 100, culture medium 20 can be removed and replaced by a different solution, which eliminates feeders 1 from image background.

In the setting, microbeads 10 float upwards via gravity and stay away from single cell 100. Feeder 1 are retained by microbeads 10. The distance between single cell 100 and feeders 1 can be controlled by adjusting the volume of culture medium 20. If extended culture is required, fresh feeders 1 from flask 30 can be used to revive the supportive strength of feeders to single cell 100 in microwell 130.

In prior art, feeders form a monolayer on bottom of wells. The bottom of a microwell is dramatically reduced so that there is no much bottom surface available for feeders. Microbeads in the invention provides a 3-dimensional structure for feeders to grow, which allows a setting of a large quantity of feeders into a tiny microwell to support the single cell.

Although the description above contains specifications. It will apparent to those skilled in the art that a number of other variations and modification can be made in this invention without departing from its spirit and scope. Microbeads 10, for example, can be made solid and heavier than water. The shape of microbeads 10 can be irregular instead of sphere. The material of microbeads 10 can be porous to further increase surface area for attachment of feeders. Therefore, the description as set out above should not be constructed as limiting the scope of the invention but as merely providing illustration of one of the preferred embodiments of the invention.

1. A method for manipulating feeders in a culture medium to support a co-culture in wells, comprising;
a utilization of water insoluble micromembers, being smaller than said wells and movable within said culture medium, to provide favorable surfaces for attachment and growth of said feeders, to carry said feeders into said wells, to keep said feeders at desired location in said wells, and to remove said feeders.

2. The method of claim 1 wherein said micromembers are hollow glass microbeads.

3. The method of claim 1 wherein said feeders locate in top region of said culture medium.

4. A method of a co-culture in wells,
comprising;
having cells in said wells immersed in a culture medium;
having feeders capable of conditioning said culture medium;
having microbeads with favorable surfaces for attachment and growth of said feeders; and
using said microbeads to retain said feeders at a suitable location in said wells to condition said culture medium for supporting said cells during said co-culture.
